(19) United States
(12) Patent Application Publication
(10) Pub. No.: US 2008/0071331 A1
(43) Pub. Date: Mar. 20, 2008

(54) Photodynamic Therapy For The Treatment Of Prostatic Conditions

(75) Inventors: Valery Rubinchik, Richmond (CA); Mitchell D. Visser, Burnaby (CA)

Correspondence Address: MORRISON & FOERSTER LLP, 12531 HIGH BLUFF DRIVE, SUITE 100, SAN DIEGO, CA, 92130-2040, US

(73) Assignee: QLT Inc., Vancouver (CA)

(21) Appl. No.: 10/589,086

(22) PCT Filed: Feb. 13, 2004
(86) PCT No.: PCT/CA04/00205

§ 371 Date: Jul. 6, 2007

(51) Int. Cl. A61N 5/067; A61M 29/02; B65D 69/00

(52) U.S. Cl. 607/89; 206/223; 604/96.01

(57) ABSTRACT

The present invention relates to a photodynamic method of treating prostatic disorders such as BPH. The method comprises: administering a photosensitizer by direct injection to the prostate tissue of a subject afflicted with or suspected of being afflicted with a prostatic disorder; and irradiating the prostate tissue with energy at a wavelength appropriate to activate the photosensitizer, wherein a guidewire is utilized to position the injection device and the irradiation apparatus.

### FIELD OF THE INVENTION

This invention relates to a method of treating prostatic diseases with photodynamic therapy (PDT). The use of PDT and appropriate photosensitizers for treating prostatic conditions, especially benign prostatic hyperplasia (BPH), is contemplated and disclosed.

### BACKGROUND OF THE INVENTION

The prostate is the organ of the body most often affected by disease in men past middle age. The most common condition to affect the prostate is benign prostatic hyperplasia (BPH). Histological evidence of BPH is found in 80% of men over the age of 60. BPH is characterised by a gradual increase in glandular and fibromuscular tissue of the prostate. The proliferation of prostatic tissue associated with BPH can lead to a narrowing or occlusion of the urethra which, in turn, can cause urine outflow obstruction. The disease generally progresses very slowly and symptoms are often mild enough that watchful waiting is the recommended course of action. However, the condition can have serious consequences such as hydronephrosis and renal failure.

There are both medicinal and surgical options for the treatment of BPH. The current therapies are designed to relieve the narrowing of the urethra either through medications or surgery.

Medications include 5-α reductase inhibitors (such as finasteride) and α-adrenoceptor blockers (such as prazosin, terazosin, doxazosin and tamsulosin). Although both classes of drug provide relief of symptomatic BPH the effect wanes over the long-term. In addition, the medications must be taken daily and can have side effects such as dizziness, postural hypotension, ejaculatory dysfunction, decreased libido and impotence.

Surgical therapies for BPH include insterstitial laser coagulation of the prostate (ILC), transurethral microwave thermotherapy (TUMT) which uses microwaves to heat and destroy excess tissue, transurethral needle ablation (TUNA) which uses low-level radiofrequency energy to ablate a defined region of the prostate, and transurethral resection of the prostate (TURP) which is the most commonly used surgical treatment for BPH. TURP involves using an instrument passed through the urethra to remove prostate tissues that surround the urethra. While the current surgical remedies all have reasonable therapeutic outcomes they also have drawbacks. For example, the current therapies are often invasive and can cause damage to tissues other than the target tissue. Also, the surgical procedure can be lengthy, painful, have long recovery periods, necessitate prolonged catheterisation, and require careful monitoring including the use of a rectal probe.

Photodynamic therapy (PDT) has been proposed as one alternative for treating prostatic tissue. U.S. Pat. No. 5,514,669 (Selman) describes a method of treating the symptoms associated with BPH or prostatitis comprising sensitizing the prostatic tissue with an effective amount of photosensitive composition which accumulates in the tissue and exposing the sensitized tissue to a light energy source whereby the photosensitive composition absorbs the light or undergoes a photochemical reaction. The paper entitled “Studies of Tin Ethyl Etiopurpurin Photodynamic Therapy of the Canine Prostate” Journal of Urology, Vol. 165, 1795-1801 (May 2001), Selman et al. described PDT of Canine prostate after a slow bolus intravenous injection of 0.5-1.0 mg/kg of SnET2. Another canine study is detailed in “Photodynamic Therapy in the Canine Prostate Using Motexafin Lutetium” Clinical Cancer Research, Vol. 7, 651-660 (March 2001), His et al. In this study motexafin lutetium was administered to the dogs by intravenous injection.

### SUMMARY OF THE INVENTION

The present disclosure relates to a photodynamic method of treating non-cancerous prostatic disorders such as BPH or prostatitis. In one embodiment, the method comprises: delivering photosensitizer directly into prostatic tissue of a patient suffering from or suspected of suffering from benign prostatic hyperplasia; and irradiating the prostatic tissue with a light at a wavelength appropriate to activate the photosensitizer. Frequently, the photosensitizer is delivered directly to the prostate such that the peak concentration of photosensitizer in the prostate is at least 3 mm from the urethral wall.

Although the present methods have been found useful in non-cancerous prostatic disorders it is believed that the method would have utility for treating cancerous or pre-cancerous prostatic disorders such as prostatic intraepithelial neoplasia (PIN), prostate cancer, and others. Frequently, the present methods may be used to treat cancerous or pre-cancerous prostatic disorders that are not amenable with surgery. The present methods may be used alone or in conjunction with other therapies. In an often included embodiment, the present methods may be used to treat subjects who have previously been treated for cancerous or pre-cancerous prostatic disorders but are showing signs of recurrence (for example, through increased PSA levels, worsening prostate biopsies, etc.).

In a frequent embodiment, a method of treating cancerous or pre-cancerous prostatic disorders according to the present invention involves: delivering photosensitizer directly into prostatic tissue of a subject suffering from or suspected of suffering from a cancerous or precancerous prostatic disorder; and irradiating the prostatic tissue with a light at a wavelength appropriate to activate the photosensitizer. Often the photosensitizer is delivered directly to the prostate such that the peak concentration of photosensitizer in the prostate is at least 3 mm from the urethral wall.

In another frequent embodiment, the present disclosure also relates to a method of delivering a photosensitizer directly to the prostate such that the peak concentration of photosensitizer in the prostate is at least 3 mm from the urethral wall. Also frequently, the present disclosure relates to a method of delivering a photosensitizer directly to the prostate by injecting photosensitizer into the prostate at least 3 mm from the urethral wall. For example, between about 3 mm to about 25 mm from the wall of the urethra.

In a frequent embodiment, the photosensitizer is administered such that the peak concentration in the prostate is a sufficient distance from the urethra so that diffusion of the photosensitizer towards the urethra results in tissue concentrations immediately adjacent to the urethra that are insufficient to cause an adverse photodynamic reaction. Also frequently, the peak concentration of photosensitizer in the prostate is a sufficient distance from the prostatic capsule so that light absorbed by the photosensitizer prevents the light from reaching periprostatic tissues beyond the prostatic capsule.

The present disclosure also often relates to a device for delivering a photosensitizer directly to the prostate such that the peak concentration of photosensitizer in the prostate is at least 3 mm from the urethral wall and to a device for injecting a photosensitizer into the prostate at least 3 mm from the urethral wall. Often the photosensitizer is delivered such that its peak concentration is between about 3 mm and about 25 mm, and/or between about 5 mm to about 20 mm, and/or about 7 mm to about 15mm.

The present description relates to methods and kits useful for treating prostatic disorders such as BPH or prostatitis. In a frequent embodiment, a method for treating a prostatic disorder is provided comprising: directly administering a photosensitizer, preferably by direct injection, to the prostate tissue of a subject afflicted with or suspected of being afflicted with a prostatic disorder; and irradiating the prostate tissue with energy at a wavelength appropriate to activate the photosensitizer, wherein a guidewire is utilized to position the photosensitizer delivery device and the irradiation apparatus. In a frequent aspect, the photosensitizer is administered utilizing delivery device that comprises a channel for insertably receiving a guidewire therethrough. The delivery device can be an injection device. The injection device often comprises an injection element and a channel for insertably receiving a guidewire therethrough. The activation energy is delivered utilizing an irradiation apparatus comprising an energy source and a channel for insertably receiving a guidewire therethrough. Frequently, the guidewire is inserted or introduced in the subject before either the injection device or the irradiation apparatus. Also frequently, the guidewire is removed from the subject prior to the irradiating step.

In a frequent embodiment, the irradiation apparatus comprises an inflatable anchor chamber and/or an inflatable treatment chamber. Often, the inflatable anchor chamber and the inflatable treatment chamber comprise separate elements.

In another frequent embodiment, the photosensitizer delivery device is compatible with a cystoscope that permits the insertable positioning of the delivery device within the urethra of the subject and adjacent to the prostate tissue. Also frequently, the guidewire comprises a proximal and distal end and the distal end is pre-positioned in the bladder of the subject prior to slidably inserting the inflatable member over the guidewire. Often the cystoscope and the guidewire are removed from the subject prior to the irradiation step.

In an often included embodiment, the activation energy source comprises a proximal and distal end, and further comprises a fiber optic member having a light emitting diffuser situated on the distal end of the fiber optic member. Frequently, the light emitting diffuser is aligned with the treatment chamber prior to irradiating the treatment area. Often the activation energy is delivered by means of a laser, a fibre optical illumination device, and/or combination thereof. In a frequent embodiment, the activation energy is delivered transurethrally.

In a frequent embodiment, the photosensitizer is delivered by transurethral injection into the prostate. Further, often the photosensitizer is selected from pro-porphyrins, porphyrins, and/or mixtures thereof. Frequently, the photosensitizer comprises a green porphyrin.

In another often included embodiment a catheter device is provided of the type for treating prostatic disorders in a subject utilizing PDT, the improvement comprising: a guidewire for positioning the catheter device in the subject to perform the PDT, wherein the catheter is slidably positioned for the delivery of energy to the prostate tissue of the subject over the guidewire, and after the guidewire is pre-positioned in the subject. Frequently, the catheter comprises a balloon type catheter. Also frequently, the energy comprises an activation energy capable of activating a photosensitizer. Often the photosensitizer is administered to the subject such that it accumulates in the prostate tissue of the subject, for example, by direct injection to the prostate.

The present description further provides kits. In a frequent embodiment, a kit is provided for treating prostatic disorders, comprising: a guidewire; a means for a localized introduction of a photosensitizing agent to prostate tissue in a subject to create a treatment area; an irradiating member for irradiating the treatment area with a energy at a wavelength appropriate to activate the photosensitizing agent, the irradiating member having a central lumen for slidably receiving the guidewire; and a cystoscope having a central lumen for slidably receiving the guidewire and/or the means for localized introduction of a photosensitizing agent therethrough. On occasion, the presently contemplated kits may contain one or more, including particular combinations, of these aspects.

### BRIEF DESCRIPTION OF THE DRAWINGS

FIG. 1 shows an activation energy delivery device.

FIG. 2 show an activation energy delivery device in vivo.

### DETAILED DESCRIPTION OF THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms.

As used herein, “afflicted” as it relates to a disease or disorder refers to a subject having or directly affected by the designated disease or disorder.

As used herein, the term “subject” is not limited to a specific species or sample type. For example, the term “subject” may refer to a patient, and frequently a human patient. However, this term is not limited to humans and thus encompasses a variety of mammalian species.

As used herein, the term “delivered directly” refers to non-systemic methods of administering photosensitizer to the target tissue.

As used herein, the term “prostatic disorder” refers to any of a variety of disorders or diseases affecting prostate tissue in a subject. In one aspect, a prostatic disorder refers to acute or chronic prostatitis, benign prostatic hyperplasia (BPH), prostatic intraepithelial neoplasia, and/or prostate cancer. Frequently, the prostatic disorder comprises BPH.

As used herein, the term “cystoscope” refers to a tubular apparatus for examining the interior of the urethra and/or bladder. Often a cystoscope of the present disclosure comprises a modified cyctoscope configured to work together with the various aspects of the invention described herein.

As used herein, the term “inflatable chamber” or “inflatable balloon” refers to a balloon-type apparatus or catheter as further described herein.

As used herein, the phrase “adjacent to the prostate tissue” refers to an area next to or adjoining prostate tissue. Often the prostate tissue is in a subject. In one aspect, this phrase refers to an area within the urethra of a subject that is bordered by prostate tissue.

As used herein, the term “anchor chamber” or “anchoring balloon” refers to a chamber that, anchors the device in a position to enable the delivery of treatment. Frequently, the anchor chamber is slidably positioned within the bladder of a subject. Also frequently, the anchor chamber is inflatable, and when inflated within the bladder of a subject, prohibits its removal from the bladder. When inflated, the anchor chamber frequently comprises a diameter larger than the urethral opening through which the inflatable chamber passed to enter the bladder. When the anchor chamber is inflated, it may be inflated with a gas or fluid.

As used herein, the term “treatment chamber” refers to a chamber comprised of, at least in part, transparent or translucent material. Frequently, the chamber comprises an inflatable chamber. However, the treatment chamber need not be inflatable to function in accordance with the present methods and kits. Also frequently, a treatment chamber refers to a chamber from which activation energy is produced/emitted. When the treatment chamber is inflated, it may be inflated with a gas or fluid.

As used herein, “photosensitizer” or “photosensitizing agent” means a chemical compound which, when contacted by radiation, induces changes to, or destruction of, the prostatic tissue. Preferably, the chemical compound is nontoxic to humans or is capable of being formulated in a nontoxic composition. Preferably, the chemical compound in its photodegraded form is also nontoxic. A listing of photosensitive chemicals may be found in Kreimer-Birnbaum, Sem. Hematol. 26:157-73, 1989 (incorporated herein by reference) and in Redmond and Gamlin, Photochem. Photbiol. 70 (4): 391-475 (1999). The invention may be practiced with a variety of synthetic and naturally occurring photosensitizers, including, but not limited to, pro-drugs such as the pro-porphyrin 5-aminolevulinic acid (ALA) and derivatives thereof, porphyrins and porphyrin derivatives e.g. chlorins, bacteriochlorins, isobacteriochlorins, phthalocyanine and naphthalocyanines and other tetra- and poly-macrocyclic compounds, and related compounds (e.g. pyropheophorbides, sapphyrins and texaphyrins) and metal complexes (such as, but not limited by, tin, aluminum, zinc, lutetium). Tetrahydrochlorins, purpurins, porphycenes, and phenothiaziniums are also within the scope of the invention. Other suitable photosensitizers include bacteriochlorophyll derivatives such as those described in WO-A-97/19081, WO-A-99/45382 and WO-A-01/40232. A preferred bacteriochlorophyll is palladium-bacteriopheophorbide WST09 (Tookad™). Preferably the photosensitizers are selected from pro-porphyrins, porphyrins, and mixtures thereof. Some examples of pro-drugs include aminolevulinic acid such as Levulan™ and aminolevulinic acid esters such as described in WO-A-02/10120 and available as Metvix™, Hexvix™ and Benzvix™. Some examples of di-hydro or tetra-hydro porphyrins are described in EP-A-337,601 or WO-A-01/66550 and available as Foscan™ (temoporfin).

In particular embodiments it is preferred that the photosensitizers are selected from those which photobleach upon exposure to activation energy.

In frequent embodiments of the disclosure, the photosensitizer is selected from a particularly potent group of photosensitizers known as green porphyrins, which are described in detail in U.S. Pat. No. 5,171,749 (incorporated herein by reference). The term “green porphyrins” refers to porphyrin derivatives obtained by reacting a porphyrin nucleus with an alkyne in a Diels-Alder type reaction to obtain a mono-hydrobenzoporphyrin. Such resultant macropyrrolic compounds are called benzoporphyrin derivatives (BPDs), which is a synthetic chlorin-like porphyrin with various structural analogues, as shown in U.S. Pat. No. 5,171,749. Typically, green porphyrins are selected from a group of tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). Metallated forms of a Gp, in which a metal cation replaces one or two hydrogens in the center of the ring system, may also be used in the practice of the invention. The preparation of the green porphyrin compounds useful in this invention is described in detail in U.S. Pat. No. 5,095,030 (hereby incorporated by reference).

Frequently, the BPD comprises a benzoporphyrin derivative diester di-acid (BPD- DA), mono-acid ring A (BPD-MA), mono-acid ring B (BPD-MB), or mixtures thereof. These compounds absorb light at about 692 nm wavelength and have improved tissue penetration properties. The compounds of formulas BPD-MA and BPD-MB may be homogeneous, in which only the C ring carbalkoxyethyl or only the D ring carbalkoxyethyl would be hydrolyzed, or may be mixtures of the C and D ring substituent hydrolyzates. A number of other BPD B-ring derivatives may also be used in the present methods. These derivatives have the following general formula:

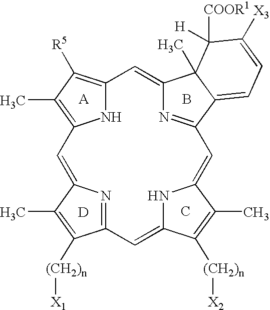

wherein; R⁵ is vinyl, R¹ and R⁶ are methyl, and n is 2. X₁, X₂, and X₃ are listed in the tables below;

TABLE 1
Hydrophilic BPD B-ring analogs

| Drug | X₁ | X₂ | X₃ |
|---|---|---|---|
| QLT0061 | COOH | COOH | COOH |
| QLT0077 | CONH(CH₂)₂N⁺(CH₃)₃I⁻ | CONH(CH₂)₂N⁺(CH₃)₃I⁻ | COOCH₃ |
| QLT0079 | CONH(CH₂)₂N⁺(CH₃)₂((CH₂)₃CH₃ | CONH(CH₂)₂N⁺(CH₃)₂((CH₂)₃CH₃) | COOCH₃ |
| QLT008 | CONHCH(COOH)CH₂COO | CONHCH(COOH)CH₂COOH | COOCH₃ |
| QLT0092 | CONH(CH₂)₂NH(CH₃)₂CF₃COO⁻ | CONH(CH₂)₂NH(CH₃)₂CF₃COO− | COOCH₃ |
| QLT009 | CONHCH₂COOH | CONHCH₂COOH | CONHCH₂COOH |

TABLE 2
Lipophilic BPD B-ring analogs

| Drug | X1 | X2 | X3 |
|---|---|---|---|
| QLT0060 | CO(O(CH₂)₂)0H | CO(O(CH₂)₂)0H | COOCH₃ |
| QLT0069 | COOCH₃ | COOCH₃ | COOH |
| QLT0078 | CO(O(CH₂)₂)₂0H | CO(O(CH₂)₂)₂0H | COOCH₃ |
| QLT0080 | CO(O(CH₂)₂)₃OH | CO(O(CH₂)₂)₃OH | COOCH₃ |
| QLT0081 | CO(O(CH₂)₂)₂OCH₃ | CO(O(CH₂)₂)₂OCH₃ | CO(O(CH₂)₂)₂OCH₃ |
| QLT0082 | CO(O(CH₂)₂)₂OH | CO(O(CH₂)₂)₂OH | CO(O(CH₂)₂)₂OH |
| QLT0083 | CO(O(CH₂)₂)₃OH | CO(O(CH₂)₂)₃OH | CO(O(CH₂)₂)₃OH |
| QLT0087 | CO(O(CH₂)₂)₄OH | CO(O(CH₂)₂)₄OH | COOCH₃ |
| QLT0088 | COOCH₃ | COOCH₃ | CONH(C₆H₄)(C₅H₁₀N) |
| QLT0090 | CO(O(CH₂)₂)₅OH | CO(O(CH₂)₂)₅OH | COOCH₃ |
| QLT0093 | CO(O(CH₂)₂)₅OH | CO(O(CH₂)₂)₅OH | CO(O(CH₂)₂)₅OH |

Preferred photosensitizers are the benzoporphyrin derivative mono-acid (BPD-MA), lemuteporfin (also known as QLT0074 & set forth in U.S. Pat. No. 5,929,105 as A-EA6) and B3 (as set forth in U.S. Pat. No. 5,990,149). Most frequently the photosensitizer comprises QLT0074 which has the structure:

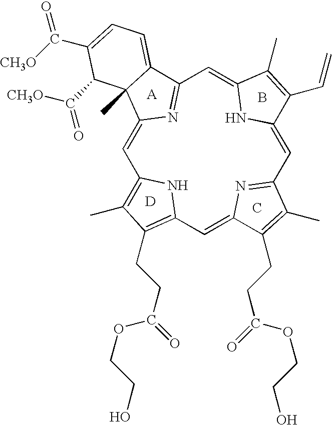

Additionally, the photosensitizers used in the invention may be conjugated to various ligands to facilitate targeting. These ligands include receptor-specific peptides and/or ligands as well as immunoglobulins and fragments thereof. Preferred ligands include antibodies in general and monoclonal antibodies, as well as immunologically reactive fragments of both.

Dimeric forms of the green porphyrin and dimeric or multimeric forms of green porphyrin/porphyrin combinations can be used. The dimers and oligomeric compounds of the invention can be prepared using reactions analogous to those for dimerization and oligomerization of porphyrins per se. The green porphyrins or green porphyrin/porphyrin linkages can be made directly, or porphyrins may be coupled, followed by a Diels-Alder reaction of either or both terminal porphyrins to convert them to the corresponding green porphyrins. Of course combinations of two or more photosensitizers may be used in the practice of the invention.

In addition to the above mentioned preferred photosensitizing agents, other examples of photosensitizers useful in the present methods, devices and kits include, but are not limited to, green porphyrins disclosed in U.S. Pat. Nos. 5,283,255, 4,920,143, 4,883,790, 5,095,030, and 5,171,749; and green porphyrin derivatives, discussed in U.S. Pat. Nos. 5,880,145 and 5,990,149. Several structures of typical green porphyrins are shown in the above cited patents, which also provide details for the production of the compounds.

The present methods frequently involve the photodynamic treatment of prostatic disorders. It is preferred that the present methods be used for treating prostatic disorders, often non-cancerous prostatic disorders. The present methods are especially useful in treating BPH. See, e.g., CA 2,418,937.

In one embodiment, the methods involve the administration of photosensitizer to prostatic tissue of a patient afflicted with, or suspected of afflicted with, a prostatic disorder. For example, the patient may be afflicted with BPH. The photosensitizer is directly delivered to the prostate such that the peak concentration of photosensitizer is at least 3 mm from the wall of the urethra.

The presently contemplated photosensitizers are frequently directly delivered to the prostate such that the peak concentration of photosensitizer is located at a sufficient distance from the urethra so that diffusion of the photosensitizer towards the urethra results in tissue concentrations immediately adjacent to the urethra or in the urethra itself that are insufficient for adverse PDT reactions. Often, the peak concentration of photosensitizer is at least about 5 mm, more preferably at least about 7 mm, even more preferably at least about 10 mm, away from other tissues such as the bladder neck or urinary sphincter. Furthermore, frequently the peak concentration of photosensitizer is also located at a sufficient distance from the prostatic capsule so that light absorbed by the photosensitizer prevents the light from reaching periprostatic tissues beyond the prostatic capsule.

It has been found that delivering the photosensitizer in such a manner reduces the incidence of photodynamic damage to non-target tissues such as the urethral lumen or healthy prostatic tissue.

While not wishing to be bound by theory, it is believed that the use of photodynamic therapy for treating BPH is advantageous because the photosensitizers selectively accumulate in hyperproliferative tissues and, consequently, better target the hyperplastic prostate cells. Also, the fact that both light and drug are required for a photodynamic effect enables more accurate targeting of the hyperplastic tissue. The present methods may be used alone or in conjunction with other therapies.

It has also been found that in certain embodiments of the present methods, the photosensitizer diffuses away from the delivery site in a relatively uniform manner which enables the physician to better control the amount of prostatic tissue that is ablated.

It has further been found that, in some embodiments, the present method creates a ‘shadow’ where the high concentration of photosensitizer at the delivery site absorbs much of the activation energy and, consequently, shields the tissue on the far side of the prostate from excessive photodynamic damage.

Moreover, it has been found that the present methods and devices are particularly useful when the patient has a American Urological Association/International Prostate Symptom Score (AUA/IPSS) of greater than 7 (see “Management of BPH”, American Urological Association (2003)).

In a frequent embodiment, the photosensitizer is delivered in such a way as to ensure that the peak concentration of photosensitizer is at least 3 mm from the wall of the urethra. Often, the peak concentration of photosensitizer is from 3 mm to about 25 mm from the wall of the urethra. More frequently the peak concentration of photosensitizer is from about 5 mm to about 20 mm from the wall of the urethra. Even more frequently the peak concentration of photosensitizer is from about 7 mm to about 15mm from the wall of the urethra.

The site of peak concentration of photosensitizer is usually the site of delivery. Therefore, the photosensitizer is often delivered directly into the prostate at least 3 mm from the wall of the urethra. More frequently, the photosensitizer is delivered directly into the prostate from 3 mm to about 25 mm from the wall of the urethra. Also frequently the photosensitizer is delivered directly into the prostate from about 5 mm to about 20 mm from the wall of the urethra. Often the photosensitizer is delivered directly into the prostate from about 7 mm to about 15 mm from the wall of the urethra. Frequently, the photosensitizer is delivered directly into the prostate about 10 mm from the wall of the urethra.

Frequently, the prostate injections are at least about 5 mm, more preferably at least about 7 mm, even more preferably at least about 10 mm away from other tissues such as the bladder neck or urinary sphincter.

Any suitable photosensitizing agent or mixture of agents may be used herein. Frequently, these will absorb radiation or activation energy in the range of from 400 nm to 800 nm, typically from 600 nm to 750 nm.

The photosensitizer can be delivered by any suitable route of administration. For example, transabdominal, transarterial, transrectual, transperineal, or transurethral approaches may be used. The photosensitizer is most frequently delivered via a transurethral or transrectal approach. More frequently the photosensitizer is delivered via a transurethral approach.

One preferred method of delivering the photosensitizer is by injection. Any suitable injection device may be used. For example, a conventional cytoscope with a needle. Suitable cytoscopes will be familiar to those skilled in the art and include ones available from Karl Storz Endoskope GmbH (Königin-Elisabeth-Str. 60, Berlin, DE), Olympus America Inc. (2 Corporate Center Drive, Melville, N.Y., USA), ACM1 (136 Turnpike Road, Southborough, Mass., USA), Richard Wolf GmbH (Postfach 1164, Knittlingen, DE), among others.

In order to provide for a good spread of the photosensitizer, frequently the prostatic tissue is injected two or more times, also frequently three or more times, and often four times. It is preferred that the injection(s) are placed so as to avoid the rectum. Frequently at least one injection is done in each lobe of the prostate. Also frequently two injections are done into each lobe. The injections are often placed east-west rather than north-south.

Frequent injection devices include Transurethral Injection Devices (TID). Presently contemplated TID devices often comprise those which can be used in conjunction with conventional cytoscopes. Frequently the TID has a needle suitable for injecting the photosensitizer into the prostatic tissue. For example, the needle can be angled to enable easier injection or it can be flexible enabling the tip to move with the cytoscope. If the needle is angled it often has an angle of deflection of more than about 30°, more often by more than about 40°, even more often by more than about 50°. Especially frequent are needles having an angle of deflection of 60°. The TID also frequently has a 20 g hollow needle. Suitable TID are available from InjecTx™ (San Jose, Calif., USA).

The injection device may have one or more defining characteristics. In an occasional embodiment the injection device comprises a device having a retractable needle such that upon appropriate or pre-designated placement of the injection device, a needle is protracted from the device and inserted into a target tissue, e.g., the prostate tissue. Often the needle is protracted such that it does not extend beyond the distal end of the injection device. In a frequent embodiment, the needle is retracted into the body of the injection device after the delivery of a photosensitizer to a target tissue. Often the needle is retracted into the body of the injection device after one or more injections into a target tissue. In a frequent embodiment, the needle aspect of the injection device is retracted during the positioning of the injection device in a subject and/or removal therefrom.

In a further occasional embodiment, the injection device comprises a means for delivery of a photosensitizer to a target tissue that becomes exposed upon contact of the injection device with a target tissue.

Frequently the needle is hollow having one opening for the delivery of photosensitizer. On occasion, the needle has one or more openings for the delivery of photosensitizer. In an occasional embodiment, means are provided that deflect the needle as it is protracted from the device at a specified angle from the injection device. In a further occasional embodiment, the needle is provided in a “pre-stressed” form such that when it is protracted from the injection device the stress in the pre-stressed needle is released such that the needle takes on a curved configuration. A frequent injection device generally incorporates a needle type apparatus capable of piercing, or being otherwise inserted into a target tissue. As one of skill in the art would appreciate, unless specifically indicated, the presently contemplated needles can take many forms not specifically limited to a hollow needle having a distal opening.

Once the photosensitizer has been delivered to the prostatic tissue it can be activated by any suitable energy source in any suitable manner. The activation energy is frequently delivered directly to the prostate.

Sufficient time is often elapses between delivery of the photosensitizer and administration of the activation energy to allow the photosensitizer to distribute within the target tissue. The exact length of time can vary according to the type of photosensitizer and the target tissue but, in general, it is preferred that at least 5 minutes, more preferably at least 10 minutes, elapses between delivery of the photosensitizer and administration of the activation energy.

Frequently, the activation energy comprises a wavelength close to at least one of the absorption peaks of the photosensitizer. This wavelength differs for different photosensitizers. For example, BPD-MA has an absorption peak at 689 nm and so, when BPD-MA is the photosensitizer used, the wavelength of the activation energy is frequently at or close to 689 nm. The photosensitizer ALA-methyl ester (available under the tradename Metvix) has an absorption peak at 635 nm and so when this photosensitizer is used the activation energy is often at or close to 635 nm. ALA (available under the tradename Levulan) has an absorption peak at 417 nm and at 630 nm so when this photosensitizer is used the activation energy is often at or close to 417 nm and/or 630 nm.

The duration of treatment is often short enough so the desired effect is achieved before the photosensitizer is washed out of the prostate. It is also preferred that the duration of treatment is short enough to avoid major discomfort to the patient. Frequently, the duration of treatment is less than about 60 mins, more often less than about 30 mins, even more often less than about 15 mins. Less occasionally, the duration of treatment may be longer than 60 minutes.

The activation energy should be capable of penetrating the tissue to a depth sufficient to activate the photosensitizer at the target tissue. In general, the longer the wavelength of the activation energy, the greater the penetration. Frequently, the activation energy penetrates at least 1 mm, more often at least 2 mm, even more often at least 3 mm through prostatic tissue. Also frequently, the activation energy penetrates to the area containing the peak concentration of photosensitizer.

The activation energy herein may be provided by any suitable means. Generally, the activation energy is provided by a light source although it is contemplated that x-ray and/or ultrasound sources may be used. Activation energy sources often include lasers, filtered full spectrum arc lamps, light emitting diodes, and/or combinations thereof. More frequently, the energy source used herein is selected from lasers, light emitting diodes, and combinations thereof. Even more frequently the energy source is a laser. Examples of suitable lasers include the 630 PDT (Diomed, Andover, Mass., USA), Ceralas™ (Biolitec AG, Winzerlaer Str.2a, Jena, DE), and the KTP/532™ or KTP/YAG™ (Laserscope, San Jose, Calif., USA).

The activation energy may be delivered to the prostate by any suitable means. As mentioned above, the activation energy is most frequently delivered directly to the prostate. Therefore, the delivery device is generally adapted or adaptable to deliver activation energy directly to the prostate. The activation energy is generally delivered via a transurethral or transrectal approach. Most frequently, the activation energy is delivered via a transurethral approach. The most frequent delivery devices are flexible to allow the physician to insert the device through a lumen, especially the urethra, to reach the prostate. Frequently the activation energy is transmitted from the energy source to the target with a fiber optical device. Suitable fiber optics include Optiguide™ Fiber Optics (Diomed, Andover, Mass., USA) and the RD light diffuser (Medlight, Ecublens, CH).

In a frequent embodiment the activation energy is delivered to the prostate by means of a balloon catheter. Some suitable catheters are described in CA-A-2,255,058 (herein incorporated by reference). The balloon catheter often comprises a treatment window which allows the passage of the activation energy to the prostate. Also frequently, the balloon catheter further comprises an anchoring balloon. Frequently, the balloon having the treatment window comprises reflective end caps.

In a frequent embodiment, a method is provided comprising a combination of one or more steps. The cystoscope or modified cystoscope is frequently inserted through the urethra of a subject. Often, the distal end of the cystoscope is positioned within the bladder of the subject. Frequently, the distal end of the cystoscope is positioned within the urethra of the subject, adjacent to the prostate. Also frequently, the distal end of the cystoscope is positioned such that the injection device can be utilized to administer a therapeutic agent to the prostate tissue. As indicated, an injection device is contained within or insertably positioned within the cystoscope prior to, concurrently with or after insertion of the cystoscope. The injection device is then positioned to administer a therapeutic composition to the prostate tissue in the subject at one or more locations in the prostate. Multiple injections may be performed. The injection device is then withdrawn from the cystoscope leaving a channel within the cystoscope. Frequently, the guidewire is inserted through the channel so a portion of the guidewire is positioned in the bladder of the subject. The cystoscope is then optionally removed from the subject. An irradiation apparatus or catheter is inserted axially over the guide wire and positioned to provide activation energy to the prostate tissue. Although not intending to be bound by theory, the irradiation apparatus often comprises a catheter having an anchoring balloon and a treatment balloon that is inserted axially over the guide wire and positioned in the subject such that the anchoring balloon is positioned in the bladder of the subject. In a frequent embodiment, insertion of the apparatus over the guidewire means that the proximal (near) end of the wire is threaded into the inner lumen of the catheter and the catheter is advanced over the wire into the patient until the proximal end of the guidewire comes out of the proximal end of the catheter shaft. Often, the anchor chamber or balloon will be positioned in the bladder of the subject; the guidewire is then removed from the subject and if the anchoring chamber is of the type that can be inflated, the anchoring chamber or balloon is inflated. After inflation of the anchoring chamber or balloon the catheter is gently positioned such that it contacts the bladder neck. The anchoring chamber or balloon is frequently of a size such that it is too large to go into the prostatic urethra when inflated. In an often included embodiment, the treatment chamber or balloon may also be inflated.

In an occasional embodiment, the placement of the injection device, the irradiation apparatus, the light source and/or the guidewire is monitored and/or guided through an imaging technique. Frequently the imaging technique is selected from an MRI, an X-ray, ultrasound, a CT scan, fluoroscopy, among other modes. Such imaging techniques may be offered to provide for accurate placement of the presently contemplated devices for optimal treatment delivery. In an occasional embodiment, means are provided on the injection device, the irradiation apparatus, the light source and/or the guidewire to ensure accurate imaging.

The amount of photosensitizer administered may vary. Often the type and concentration of the photosensitizer are accounted for when determining the appropriate dosage. Moreover, the status and type of tissue to which the photosensitizer is to be administered is often accounted for when determining the dosage and/or type of photosensitizer.

When the irradiation apparatus is of the type that is capable of being inflated when positioned in a subject, frequently the treatment balloon is inflated subsequent to the inflation and/or positioning of the anchor balloon. On occasion, the treatment balloon is inflated concurrently with the anchor balloon. Both the treatment and anchor balloons can be inflated with a gas or fluid. Although not intending to be bound by theory, as used herein a gas utilized to inflate a balloon of the present devices is considered a fluid. On occasion, the anchor and treatment balloons are inflated utilizing a different gas or fluid media in each balloon. A frequent media utilized in the treatment balloon permits activation energy to pass therethrough, with or without reflection or refraction. Media may be utilized that filters, enhances or reduces the level of activation energy emitted form the irradiation apparatus.

In an occasional embodiment, the distance between the anchoring chamber or balloon and the treatment chamber or balloon is often such that the treatment balloon window is positioned in the prostatic urethra. Frequently, the treatment window is positioned in the middle of the prostatic urethra. The fiber optic is inserted into the inner lumen of the catheter and positioned such that the emitting aspect of the fiber optic is aligned with the treatment balloon; thereafter the activation energy is initiated.

The guidewire comprises a means by which the catheter can be accurately positioned within the urethra and/or bladder of the subject. In a frequent embodiment the guidewire is compatible with the cystoscope or modified cystoscope utilized, in part, to enable the localized injection of a therapeutic composition, e.g., a photosensitizer. The guidewire is “compatible” in that it can be slidably inserted or removably positioned in the inner lumen or channel of the cystoscope. Often the guidewire is capable of passing entirely through the inner lumen or channel of the cystoscope with minimal modification or adjustment. In such an embodiment, the guidewire can be threaded through the cystoscope, exiting the distal end of the cystoscope and entering the bladder of the subject. Frequently the guidewire is of the type produced by Lake Region Manufacturing Co. (Chaska, Minn.), Olympus Co., among other manufacturers of guidewire devices.

The irradiation apparatus is generally compatible with the guidewire in that the apparatus is capable of being inserted over the guidewire and slidably positioned within the urethra of the subject. As such, the apparatus comprises a lumen or channel that can fit axially around and slide laterally over the guidewire.

Balloon catheters of the present description frequently comprise a treatment balloon. The treatment balloon comprises a balloon that takes any of a variety of shapes upon inflation, but frequently comprises a cylinder that extends axially around the central channel of the catheter. The treatment balloon often incorporates a treatment window through which light energy escapes the catheter and contacts the tissue surrounding the treatment balloon. The treatment window may or may not be a formally designated or defined aspect of the treatment balloon. In a frequent embodiment, the treatment window comprises a defined aspect of the treatment balloon. The treatment window on occasion may comprise a specific section of the balloon that permits the escape of light energy. Often the treatment window comprises a cylindrical section of the balloon such that the portion of the balloon not comprising the treatment window comprises the ends of the treatment balloon. Often the treatment balloon incorporates a reflective or refractive end cap such that light energy is prohibited from escaping from the treatment balloon in the direction of the bladder of the subject. Often the treatment balloon incorporates a reflective or refractive end cap on each end of a cylindrical treatment balloon. In another aspect, the treatment balloon can incorporate means that enable or enhance the scattering of light energy exiting from the treatment balloon. Frequently such means may comprise light scattering material known in the art. The light scattering material can be incorporated in any of the variety of aspects of the treatment balloon through which light energy is intended to pass, including the means (i.e., fluid) utilized to inflate the treatment balloon.

As indicated elsewhere herein, the irradiation apparatus need not comprise one or more inflatable chambers. Frequently, in such embodiments, each of the other aspects and qualities of the irradiation apparatus are similarly situated to those of the inflatable type of apparatus.

The present disclosure further contemplates kits for use in monitoring and/or treating prostatic disorders. Such kits may incorporate any or all of the aspects and components described herein. Generally the use of these kits is in accordance with the methods set forth herein, although variations of the present methods are contemplated. Further, kits of the present disclosure often contain instructions for their use.

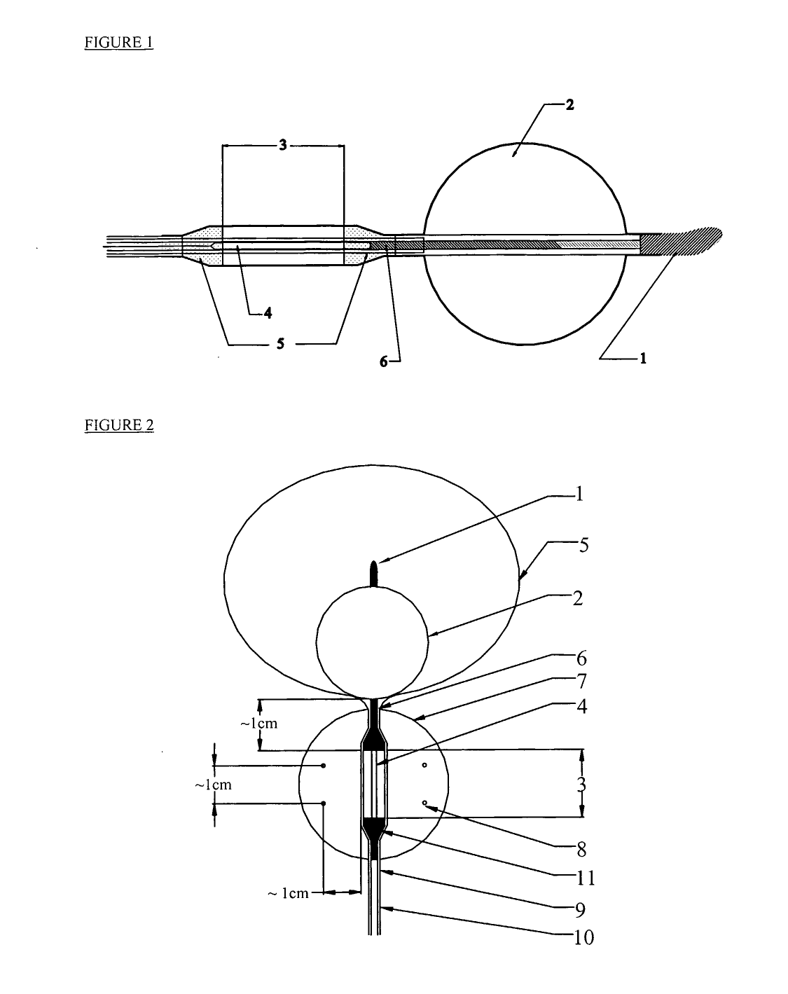

A frequent device for delivery of the activation energy is shown in FIG. 1. The device has a flexible tip (1) and an anchoring balloon (2). There is also a treatment window (3) that allows delivery of the activation energy to the prostate. The device also includes a fiber optic diffuser (4), reflective end-caps (5), and a fiber optic stopper (6).

FIG. 2 shows the device of FIG. 1 being used in a transurethral approach. The device (11) is guided via the urethra (10) and the urethral sphincter (9) using the flexible tip (1) until the anchoring balloon (2) is past the bladder neck (6). Once in the bladder (5) the balloon (2) is inflated. In FIG. 2, the distance between the anchoring balloon (2) and the treatment window (3) is approximately 1 cm. This usually puts the treatment window in line with the prostate (7). In this figure, the prostate has been injected (8) twice in each lobe approximately 1 cm from the urethra and the injection sites are also separated by approximately 1 cm. Once the device (11) is in position the activation energy can be delivered via a fiber optic diffuser (4) which is positioned such that the activation energy can exit via the treatment window.

A regimen often utilized according to the present disclosure comprises: First, administering photosensitizer directly to the prostate by transurethral injection. Preferably subjects receive one injection into the prostate on either side of the urethra. The preferred injection depth is 10 mm and it is preferred that the injections are at least 10 mm away from other tissues such as the bladder neck or urinary sphincter. The preferred photosensitizer is QLT0074 and the preferred total dose is 0.4 mg. And, second, administering activation energy via a transurethral balloon catheter and fiber optic diffuser. Preferably, the activation energy is administered approximately 30 minutes after the injections. The fiber optic diffuser preferably has reflective end caps. If light is the activation energy used it is preferred that a dose between 15 and 200 J/cm² is delivered. More preferred light doses include 25, 20, 80, 120, or 150 J/cm².

### EXAMPLE

It will be understood that the following embodiments of the present invention are intended to be illustrative of some of the possible applications or principles. Various modifications may be made by the skilled person without departing from the true spirit and scope of the invention.

Eight mature, intact, mixed-breed dogs are premedicated with butorphanol (0.4 mg per kilogram of body weight), acepromazine (0.1 mg/kg), and atropine (0.4 mg/kg), all given by intramuscular injection. Anesthesia is induced with intravenous thiopental sodium (8 mg per 0.4536 kilograms of body weight) and maintained with inhaled isoflurane and oxygen (dosage as required for anesthetic effect).

Lemuteporfin for injection (A-EA6 in U.S. Pat. No. 5,929,105) is reconstituted with Water for Injection to give a stock concentration of 2.0 mg/ml and diluted with 5% Dextrose in water to a concentration 0.2 mg/ml. A standard cystoscope is inserted into the urethra. Injections of the drug are then delivered to the prostate using a commercially available transurethral injection device (InjecTx™, San Jose, Calif., USA) which is compatible with the cystoscope. Two injections of 0.5 ml are given to each left lobe of the prostate and 2 injections of 1.0 ml are given to each right lobe of the prostate. The transurethral injection device is then removed and a guidewire is inserted through the working channel of the cystoscope.

The activation-energy delivery device comprises: a cylindrical fibre optic with a 200 μm diameter core, a 25 mm long diffuser, and a power rating of 175 mW/cm (maximum power load, 437.5 mW); a reflective-cap fibre-centering balloon catheter with a 20 mm treatment window supplied with a guidewire for positioning the device in the urethra; and a 3 W 690 nm red-light diode laser (AOC, South Plainfield, N.J., USA). The catheter is inserted and positioned using the guidewire which is fed through an internal channel of the catheter. Once the catheter is in position the balloon is inflated to secure it in place. The guidewire is removed from the catheter and the fiber optic diffuser is inserted into the catheter and secured in place.

The treatment assembly is positioned in the urethra using ultrasonic guidance. Ultrasound is also used to position the needles for four percutaneous intraprostatic injections (two per lobe separated by longitudinally by 1 cm). Each injection is at a depth of between 8 and 12 mm into the prostate. The activation energy is delivered 15 minutes after the last injection. A dose of either 25 J/cm², 50 J/cm², or 100 J/cm² is delivered.

After seven days the dogs are euthanased and the prostates were harvested. Bowel, bladder, and prostate sizes are found to be within normal ranges. The prostates all exhibit evidence of PDT-associated tissue damage in the target tissue. However, no significant PDT-associated damage is seen in the urethra.

The ordinarily skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A method of treating prostatic disorders comprising:
a) directly administering a photosensitizer to the prostate tissue of a subject afflicted with or suspected of being afflicted with a prostatic disorder; and
b) irradiating the prostate tissue with energy at a wavelength appropriate to activate the photosensitizer
wherein the photosensitizer is administered utilizing a delivery device comprising a channel for insertably receiving a guidewire therethrough, and
the activation energy is delivered utilizing an irradiation apparatus comprising an energy source and a channel for insertably receiving a guidewire therethrough, and
a guidewire is utilized to position the delivery device and/or the irradiation apparatus.

2. The method of claim 1, wherein the photosensitizer is administered by injection.

3. The method of claim 1, wherein the guidewire is used to position both the delivery device and the irradiation apparatus.

4. The method of claim 1, wherein the irradiation apparatus comprises an inflatable anchor chamber and an inflatable treatment chamber.

5. The method of claim 1, wherein the delivery device is compatible with a cystoscope that permits the insertable positioning of the delivery device within the urethra of the subject and adjacent to the prostate tissue.

6. The method of claim 1, wherein the guidewire is removed from the subject prior to the irradiating step.

7. The method of claim 1, wherein the prostatic disorder is benign prostatic hyperplasia.

8. The method of claim 1, wherein the guidewire comprises a proximal and distal end and the distal end is pre-positioned in the bladder of the subject prior to slidably inserting the channel over the guidewire.

9. The method of claim 5, wherein the cystoscope and the guidewire are removed from the subject prior to irradiation.

10. The method of claim 4, wherein the light source comprises a proximal and distal end, and a fiber optic member having a light emitting diffuser situated on the distal end of the light source.

11. The method of claim 10, wherein the light emitting diffuser is aligned with the treatment chamber prior to irradiating the treatment area.

12. The method of claim 1, wherein the peak concentration of photosensitizer is from 3 mm to about 25 mm from the wall of the urethra.

13. The method of claim 1, wherein the peak concentration of photosensitizer is from about 5 mm to about 20 mm from the wall of the urethra.

14. The method of claim 1, wherein the peak concentration of photosensitizer is from about 7 mm to about 15 mm from the wall of the urethra.

15. The method of claim 1, wherein the photosensitizer is delivered by transurethral injection into the prostate.

16. The method of claim 1, wherein the photosensitizer is selected from the group consisting of pro-porphyrins, porphyrins, and mixtures thereof.

17. The method of claim 16, wherein the photosensitizer is selected from the group consisting of green porphyrins.

18. The method of claim 1, wherein the light energy is delivered by means of a laser, a fibre optical illumination device, or combination thereof.

19. The method of claim 1, wherein the light energy is delivered transurethrally.

20. The method of claim 1, wherein the photosensitizer is injected directly into the prostate tissue such that the peak concentration of photosensitizer in the prostate is at least 3 mm from the urethral wall.

21. The method of claim 1, wherein the photosensitizer is injected into the prostate tissue at least 3 mm from the urethral wall.

22. The method of claim 1, wherein the photosensitizer is administered such that the peak concentration in the prostate is a sufficient distance from the urethra so that diffusion of the photosensitizer towards the urethra results in tissue concentrations immediately adjacent to the urethra that are insufficient to cause an adverse photodynamic reaction.

23. The method of claim 1, wherein the peak concentration of photosensitizer in the prostate is a sufficient distance from the prostatic tissue so that light absorbed by the photosensitizer prevents the light from reaching periprostatic tissues.

24. A method of treating prostatic disorders comprising:
a) administering a photosensitizer with a transurethral injection device to the prostate tissue of a subject afflicted with or suspected of being afflicted with a prostatic disorder; and
b) irradiating the prostate tissue with energy at a wavelength appropriate to activate the photosensitizer,
wherein the transurethral injection device comprises a channel for insertably receiving a guidewire therethrough, and
the activation energy is delivered transurethrally utilizing an irradiation apparatus comprising an energy source and a channel for insertably receiving a guidewire therethrough,
wherein the transurethral irradiation apparatus is positioned utilizing a guidewire.

25. A kit for treating prostatic disorders, comprising:
a) a guidewire;
b) a means for a localized introduction of a photosensitizing agent to prostate tissue in a subject to create a treatment area;
c) an irradiating member for irradiating a treatment area with an energy at a wavelength appropriate to activate the photosensitizing agent, the irradiating member having a central lumen for slidably receiving the guidewire; and
d) a cystoscope having a central lumen for slidably receiving the guidewire and/or the means for localized introduction of a photosensitizing agent therethrough.

26. In a method of inserting a balloon catheter for treating prostatic disorders in a subject utilizing photodynamic therapy (PDT), the improvement comprising:
positioning the balloon catheter device in the subject to perform the PDT using a guidewire,
wherein the balloon catheter is slidably positioned for the delivery of light energy to the prostate tissue of the subject over the guidewire, and after the guidewire is pre-positioned in the subject.
